SEQUENCE LISTING (15 sequences)

SEQ ID NO: 1 - DNA, 21 bp, Artificial Sequence
  PCR Primer
gtcagtatcacaggctgcgag

SEQ ID NO: 2 - DNA, 22 bp, Artificial Sequence
  PCR Primer
tgttcctagtcttacgccagtg

SEQ ID NO: 3 - DNA, 21 bp, Artificial Sequence
  PCR Primer
ggcgttgccgctctgaattgc

SEQ ID NO: 4 - DNA, 24 bp, Artificial Sequence
  PCR Primer
gagggactgagctggacaacccac

SEQ ID NO: 5 - DNA, 12 bp, Homo sapiens
catagggatgcc

SEQ ID NO: 6 - DNA, 12 bp, Homo sapiens
catagggacgcc

SEQ ID NO: 7 - DNA, 12 bp, Homo sapiens
cataggaacgtc

SEQ ID NO: 8 - DNA, 12 bp, Homo sapiens
cctagggatgcc

SEQ ID NO: 9 - DNA, 12 bp, Homo sapiens
aatagggatgcc

SEQ ID NO: 10 - DNA, 12 bp, Homo sapiens
aacgaggcccct

SEQ ID NO: 11 - DNA, 12 bp, Homo sapiens
aacgagaatgcc

SEQ ID NO: 12 - DNA, 12 bp, Homo sapiens
aacgaagcccct

SEQ ID NO: 13 - DNA, 12 bp, Homo sapiens
aacgagaacgtc

SEQ ID NO: 14 - DNA, 12 bp, Homo sapiens
catagggccccc

SEQ ID NO: 15 - DNA, 12 bp, Homo sapiens
catgaggatgcc

(19) United States
(12) Patent Application Publication
(10) Pub. No.: US 2014/0206561 A1
(43) Pub. Date: Jul. 24, 2014

(54) METHOD FOR PROVIDING INFORMATION ON ANTIDEPRESSANT THERAPEUTIC EFFECT USING SINGLE NUCLEOTIDE POLYMORPHISM

(71) Applicant: SAMSUNG LIFE WELFARE FOUNDATION, Seoul (KR)

(72) Inventors: Doh Kwan KIM, Seoul (KR); Jong Won KIM, Seoul (KR); Seon Woo KIM, Gyeonggi-Do (KR); Bernard J. CARROLL, Carmel, CA (US); Shinn Won LIM, Seoul (KR)

(73) Assignee: SAMSUNG LIFE WELFARE FOUNDATION, Seoul (KR)

(21) Appl. No.: 14/158,645

(22) Filed: Jan. 17, 2014

Related U.S. Application Data
(63) Continuation of application No. 13/202,443, filed on Sep. 21, 2011, now abandoned.

(30) Foreign Application Priority Data
Mar. 31, 2009 (KR) 10-2009-0027793

(51) Int. Cl. C12Q 1/68

(52) U.S. Cl. CPC C12Q 1/6883
USPC 506/9; 435/6.11; 536/23.2; 536/23.5; 506/16

(57) ABSTRACT

Disclosed is a method for providing information on the therapeutic effect of an SSRI antidepressant by identifying TPH2 gene polymorphism rs4760815, SLC6A4 gene polymorphism 5-HTTLPR, SLC6A4 gene polymorphism rs2066713, GAD1 gene polymorphism rs3828275, and GRIK2 gene polymorphism rs543196. Through the disclosed method, it is possible to select an antidepressant based on genetic information, to prevent the worsening or relapse of depression, and to establish customized depression treatment models which are effective in the development of customized new drugs, and appropriate for Korean people. Therefore, the base of domestic clinical trials can be expanded, which will enhance competitive power in the pharmaceutical market and preoccupy technology of drug prediction.

### CROSS-REFERENCES TO RELATED APPLICATIONS

This application is a continuation of U.S. patent application Ser. No. 13/202,443, filed Sep. 21, 2011. U.S. Ser. No. 13/202,443 is the U.S. national phase application, pursuant to 35 U.S.C. §371, of PCT/KR2009/002049, filed Apr. 20, 2009, designating the United States, which claims priority to Korean Application No. 10-2009-0027793, filed Mar. 31, 2009. The entire contents of the aforementioned patent applications are incorporated herein by this reference.

### SEQUENCE LISTING

The instant application contains a Sequence Listing which has been submitted in ASCII format via EFS-Web and is hereby incorporated by reference in its entirety. Said ASCII copy, created on Apr. 7, 2014, is named 89134CON301264_ST25.txt and is 2,919 bytes in size.

### TECHNICAL FIELD

The present invention relates to a method for providing information on the therapeutic effect of an antidepressant by using single nucleotide polymorphism (SNP), and more particularly to a method for providing information on the therapeutic effect of an SSRI antidepressant, which can be used to realize a customized treatment for an individual depressed patient.

### BACKGROUND ART

In general, in performing drug therapy by antidepressants on depressed patients, individual patients show different responses to the drugs. Accordingly, most antidepressants which are currently commercially available, have shown a treatment success rate of only about 50˜60%. Therefore, in order to improve the therapeutic effects, various research to predict treatment responses by using the genetic information of individual patients and to provide customized antidepressants for the individual patients has been conducted.

In the prior art, Stober, et al. reported that norepinephrine transporter gene NET G1287A polymorphism does not change the function of NET and is not significantly associated with major depression, bipolar disorder, schizophrenia, alcohol dependence or panic disorder [Reference: Stober G, Nothen M M, Porzgen P, et al. Systematic search for variation in the human norepinephrine transporter gene: identification of five naturally occurring missense mutations and study of association with major psychiatric disorders. Am J Med Genet. 1996; 67:523-532]. It is known that NET G1287A polymorphism is associated with the cerebrospinal fluid (CSF) concentration of 3-methoxy-4-hydroxyphenylglycol, a major norepinephrine metabolite [reference: Jonsson E G, Nothen M M, Gustaysson J P, et al. Polymorphisms in the dopamine, serotonin, and norepinephrine transporter genes and their relationships to monoamine metabolite concentrations in CSF of healthy volunteers. Psychiatry Res. 1998; 79:1-9], and with the response to methylphenidate, a drug with noradrenergic action, in attention deficit/hyperactivity disorder [reference: Jonsson E G, Nothen M M, Gustaysson J P, et al. Polymorphisms in the dopamine, serotonin, and norepinephrine transporter genes and their relationships to monoamine metabolite concentrations in CSF of healthy volunteers. Psychiatry Res. 1998; 79:1-9]. Also, Yoshida et al. previously examined the association between NET polymorphisms and antidepressant responses in Japanese patients [reference: Yoshida K, Takahashi H, Higuchi H, et al. Prediction of antidepressant response to milnacipran by norepinephrine transporter gene polymorphisms. Am J Psychiatry. 2004; 161:1575-1580]. They reported that the NET T-182C polymorphism was associated with a superior response to milnacipran, a serotonin-norepinephrine reuptake inhibitor (SNRI) antidepressant, and they also reported that the NET G1287A polymorphism was associated with the onset of the response, but not the final clinical improvement.

Pollock et al. examined 5-HTTLPR (serotonin transporter gene) polymorphism and the response to nortriptyline (tricyclic antidepressant, noradrenaline reuptake inhibitor antidepressant) in 23 patients and found no differences [reference: Pollock B G, Ferrell R E, Mulsant B H, et al. Allelic variation in the serotonin transporter promoter affects the onset of paroxetine treatment response in late-life depression. Neuropsycho-pharmacology .v2000; 23:587-590]. Tsapakis et al. reported the association between 5-HTTLPR and the response to tricyclic antidepressant treatment [reference: Tsapakis E M, Checkley S, Kerwin R W, Aitchison K J. Association between the serotonin transporter linked polymorphic region gene and response to tricyclic antidepressants. Eur Neuropsychopharmacol. 2005; 15:S26-S27].

Meanwhile, U.S. patent 60/895,649 disclosed a method for predicting the antidepressant treatment response by polymorphisms and combinations of genes (5HT2A, GRIK4, and BCL2) highly related to Citalopram drug treatment. Also, U.S. patent Ser. No. 11/867,400 disclosed a method for predicting the drug response according to the combination of genotypes of serotonin (5-HTT) and norepinephrine transporter (NET) in depressed patients.

However, since there have been no clinical models to date that can be used for predicting the treatment responses based on the combination of polymorphisms and haplotypes of genes (such as TPH2, GRIK2, GAD1 and SLC6A4) related to the treatment responses of SSRI antidepressants, the present inventors made an attempt to improve the treatment efficiency of depressed patients by predicting the responses to SSRI-based drugs through a clinical model.

### DISCLOSURE

### Technical Problem

The present invention has been made to provide a method for providing information on the therapeutic effect of an antidepressant by using single nucleotide polymorphism (SNP).

### Technical Solution

The present invention provides a method for providing information on a therapeutic effect of an SSRI antidepressant by identifying TPH2 gene polymorphism rs4760815, SLC6A4 gene polymorphism 5-HTTLPR, SLC6A4 gene polymorphism rs2066713, GAD1 gene polymorphism rs3828275, and GRIK2 gene polymorphism rs543196.

Also, the present invention provides a method for providing information on a therapeutic effect of an SSRI antidepressant by identifying TPH2 gene polymorphism H3 haplotype, SLC6A4 gene polymorphism H1 haplotype, SLC6A4 gene polymorphism 5-HTTLPR, GAD1 gene polymorphism rs3828275, and GRIK2 gene polymorphism rs543196.

Also, the present invention provides a method for providing information on a therapeutic effect of an SSRI antidepressant by identifying TPH2 gene polymorphism rs4760815, and SLC6A4 gene polymorphism rs2066713.

Also, the present invention provides a method for providing information on a therapeutic effect of an SSRI antidepressant by identifying TPH2 gene polymorphism H3 haplotype, and SLC6A4 gene polymorphism H1 haplotype.

Also, the present invention provides a kit for predicting a therapeutic effect of an SSRI antidepressant, the kit including primers for screening TPH2 gene polymorphism rs4760815, SLC6A4 gene polymorphism 5-HTTLPR, SLC6A4 gene polymorphism rs2066713, GAD1 gene polymorphism rs3828275, and GRIK2 gene polymorphism rs543196, genotyping primers, and a probe.

Also, the present invention provides a kit for predicting a therapeutic effect of an SSRI antidepressant, the kit including primers for screening TPH2 gene polymorphism H3 haplotype, SLC6A4 gene polymorphism H1 haplotype, SLC6A4 gene polymorphism 5-HTTLPR, GAD1 gene polymorphism rs3828275, and GRIK2 gene polymorphism rs543196, genotyping primers, and a probe.

Also, the present invention provides a kit for predicting a therapeutic effect of an SSRI antidepressant, the kit including primers for screening TPH2 gene polymorphism rs4760815, and SLC6A4 gene polymorphism rs2066713, genotyping primers, and a probe. Also, the present invention provides a kit for predicting a therapeutic effect of an SSRI antidepressant, the kit including primers for screening TPH2 gene polymorphism H3 haplotype, and SLC6A4 gene polymorphism H1 haplotype, genotyping primers, and a probe.

Also, the present invention provides a biomarker for predicting a therapeutic effect of an SSRI antidepressant, the biomarker including TPH2 gene polymorphism rs4760815, SLC6A4 gene polymorphism 5-HTTLPR, SLC6A4 gene polymorphism rs2066713, GAD1 gene polymorphism rs3828275, and GRIK2 gene polymorphism rs543196. Also, the present invention provides a biomarker for predicting a therapeutic effect of an SSRI antidepressant, the biomarker including TPH2 gene polymorphism H3 haplotype, SLC6A4 gene polymorphism H1 haplotype, SLC6A4 gene polymorphism 5-HTTLPR, GAD1 gene polymorphism rs3828275, and GRIK2 gene polymorphism rs543196. Also, the present invention provides a biomarker for predicting a therapeutic effect of an SSRI antidepressant, the biomarker including TPH2 gene polymorphism rs4760815, and SLC6A4 gene polymorphism rs2066713.

Also, the present invention provides a biomarker for predicting a therapeutic effect of an SSRI antidepressant, the biomarker including TPH2 gene polymorphism H3 haplotype, and SLC6A4 gene polymorphism H1 haplotype.

Hereinafter, the present invention will be described in detail.

In the present invention, as a result of research on the association between about 1400 SNPs within 79 candidate genes related to the treatment response of an antidepressant, and the treatment response to SSRI antidepressants which occupy a substantial part of the antidepressant market, it was found that 10 SNPs are significant (see Table 1).

Specifically, for the genetic information of TPH2 (Tryptophan Hydroxylase 2), SLC6A4 (Serotonin Transporter, 5-HTT), GAD1 (Glutamate Decarboxylase1, GABA synthesizing enzyme), and GRIK2 (Glutamate Receptor Ionotropic kainate 2), the therapeutic responses of SSRI antidepressants were determined.

In other words, some patients who have 10 responsive SNPs (Single Nucleotide Polymorphism) (see Table 1) within the genes of TPH2, SLC6A4, GAD1 and GRIK2, or 6 responsive haplotypes (H3, H4, and H5 of TPH2, H1 of SLC6A4, and H8 and H9 of GRIK2), showed the maximum therapeutic drug effect of 88% for SSRI (selective serotonin reuptake inhibitor, SSRI) antidepressants. Accordingly, based on a test of genetic information of TPH2, SLC6A4, GAD1 and GRIK2 of patients before drug prescription, when certain patients having the above mentioned genes were prescribed SSRI-based drugs, the maximum treatment success rate was 90% which is higher than the conventional average treatment success rate (50˜60%) for antidepressants by at least 20% or more.

The present inventors found that alleles of 5-HTT (serotonin transporter gene) are associated with an antidepressant response change for SSRIs, from a previous research (reference: Kim D K, Lim S W, Lee S, et al. Serotonin transporter gene polymorphism and antidepressant response. Neuroreport. 2000; 11:215-219).

The present inventors selected, as candidate gene variations for predicting antidepressant responses, the 5-HTTLPR (5-HTT gene-linked promoter region; NCBI GenBank, at the World Wide Web (www) ncbi.nlm.nih.gov; accession number AF117826; positions 25,584,988˜25,585,338 of chromosome 17) and intron 2 VNTR (variable number of tandem repeat; positions 25,570,101-25,570,300 of chromosome 17) of the 5-HTT gene.

The information on the above mentioned 5-HTT gene \textcircled{1}, \textcircled{2}, and \textcircled{3} is as follows.

Serotonin Transporter (5-HTT Gene)

Official Symbol: SLC6A4 and Name: solute carrier family 6 (neurotransmitter transporter, serotonin), member 4 [Homo sapiens]

Other Aliases: 5-HTT, 5HTT, HTT, OCD1, SERT, hSERT

Other Designations: 5-hydroxytryptamine transporter; 5HT transporter; Na+/Cl— dependent serotonin transporter; serotonin transporter; sodium-dependent serotonin transporter; solute carrier family 6 member 4

Chromosome: 17; Location: 17q11.1-q12

MIM: 182138

GeneID: 6532

Tryptophan Hydroxylase 2(\textcircled{1} TPH2 Gene)

Official Symbol TPH2 and Name: tryptophan hydroxylase 2 [Homo sapiens]

Other Aliases: FLJ37295, MGC138871, MGC138872, NTPH

Other Designations: neuronal tryptophan hydroxylase; tryptophan 5-monooxygenase 2

Chromosome: 12; Location: 12q21.1

Annotation: Chromosome 12, NC_{—}000012.10 (70618893 . . . 70712488)

MIM: 607478

GeneID: 121278

Glutamate Receptor, Ionotropic, Kainate 2(\textcircled{2} GRIK2 Gene)

Official Symbol GRIK2 and Name: glutamate receptor, ionotropic, kainate 2 [Homo sapiens]

Other Aliases: EAA4, GLR6, GLUK6, GLUR6, MGC74427, MRT6

Other Designations: OTTHUMP00000017949; bA487F5.1; excitatory amino acid receptor 4; glutamate receptor 6

Chromosome: 6; Location: 6q16.3-q21

Annotation: Chromosome 6, NC_{—}000006.10 (101953675 . . . 102623474)

MIM: 138244

GeneID: 2898

Glutamate Decarboxylase 1(\textcircled{3} GAD Gene)

Official Symbol GAD1 and Name: glutamate decarboxylase 1 (brain, 67 kDa) [Homo sapiens]

Other Aliases: FLJ45882, GAD, SCP

Other Designations: OTTHUMP00000041055; glutamate decarboxylase 1; glutamate decarboxylase 1 (brain, 67 kD)

Chromosome: 2; Location: 2q31

Annotation: Chromosome 2, NC_{—}000002.10 (171381446 . . . 171425907)

MIM: 605363

GeneID: 2571

The present inventors, as a first hypothesis, predicted the associations between SSRI efficacy and polymorphisms of 5-HTT, \textcircled{1}, \textcircled{2}, and \textcircled{3}. Also, the inventors compared the response rates to SSRIs by genotype combinations. In a further analysis, they analyzed combinations of genetic polymorphisms with the response to SSRIs. The present inventors selected, as SSRIs, fluoxetine, paroxetine or sertraline. These drugs are the most widely used drugs for treating depressive disorder of aged people in Korea. Then, they monitored the side effects to antidepressants in accordance with the UKU side effects rating scale (a scale for determining the side effects of patients administered with psychotropic drugs) as well as the treatment responses.

In the present invention, the patient subjects who participated in the experiments were all aged 18 years or more and were enrolled in the Clinical Trials Program of the Samsung Medical Center Geropsychiatry and Affective Disorder Clinics (Seoul, Korea). The affective disorder section of the Samsung Psychiatric Evaluation Schedule (SPES) used the Structured Clinical Interview for Diagnostic and Statistical Manual of Mental Disorders (Fourth Edition, Korean edition) [reference: First M B, Spitzer R L, Gibbon M, Williams J B W. Structured Clinical Interview for DSM-IV Axis I Disorders SCID I: Clinician Version, Administration Booklet. Washington, D.C.: American Psychiatric Press; 1997]. In the present invention, at least one family member living with the patient was interviewed so as to supplement the patient's report on symptoms, behaviors, functional levels, depressive episode periods, and recent treatment. In the present invention, the conditions of all the patient subjects who participated in the experiments, met DSM-IV criteria for major depressive episodes. Diagnoses were confirmed by a board certified psychiatrist on the basis of the SPES, case review notes, and other relevant data. The required minimum standard is a HAM-D (Hamilton depression scale) score of 15 in 17 items. If the patient subjects were administered other psychotropic drugs (psychopharmaceuticals) within 2 weeks of the research, or administered fluoxetine within 4 weeks of the research, they were excluded. In the present invention, potential subjects were excluded for pregnancy, critical medical conditions, abnormal laboratory baseline values, and unstable psychological characteristics (for example, suicidality), a record of alcohol or narcotic addiction, seizures, head trauma with loss of consciousness, neurologic illness. The experiment plan was approved by the institutional review board (IRB) of Samsung Medical Center. All participating patients were informed of this research, and signed written consent forms.

In the present invention, a total of 298 patients were subjected to the experiment. The patients were assigned to monotherapy with one SSRI (fluoxetine, paroxetine or sertraline) as an antidepressant. 239 patients were administered SSRI (fluoxetine [n=104], paroxetine [n=56] or sertraline [n=79]). Dose titration was completed within 2 weeks. Doses were titrated into the usual clinical range based on initial tolerability and side effects. The final daily median (interquartile range) dosages were 30.0 (20.0˜40.0, 20.0˜50.0) mg/day of fluoxetine, 20.0˜200.0 mg/day of paroxetine, and 75.0 (75.0˜100.0, 50.0˜100.0) mg/day of sertraline. These are typical clinical dosages in Asian populations, and they result in comparable blood drug levels in Western populations which require higher drug dosages. Plasma samples for measuring SSRI levels were obtained at the end of week 4. 1-2 mg of Lorazepam was prescribed to remove insomnia at bedtime. In the present invention, the patients were examined by a psychiatrist, who monitored their side effects by the UKU side effect rating scale at week 0 (first day), 0.5 (third day), 1, 2, 4, and 6. [reference: Lingjaerde 0, Ahlfors U G, Bech P, Dencker S J, Elgen K. The UKU side effect rating scale. A new comprehensive rating scale for psychotropic drugs and a cross-sectional study of side effects in neuroleptic-treated patients. Acta Psychiatr Scand Suppl. 1987; 334:1-100.]. The 17-item HAM-D was administered by a single trained rater every 2 weeks. The rater and genotype examiner were not informed of the hypotheses of the study and drug assignment. To maintain anonymity, a research coordinator managed the data and schedules. HAM-D and genotype data were not disclosed to the psychiatrist, and the rater was not informed of the genotype data.

In the present invention, the response to drugs was defined as a 50% or greater decrease in the HAM-D score at 6 weeks. Remission was defined as a HAM-D of less than 8 at 6 weeks [reference: Keller M B. Past, present, and future directions for defining optimal treatment outcome in depression: remission and beyond. JAMA. 2003; 289:3152-3160.].

The present invention shows the importance of interracial comparative analysis to verify a pharmacogenetic candidate marker. At least some of the individual variations in the antidepressant treatment outcome have a genetic basis [reference: Srisurapanont M. Response and discontinuation rates of newer antidepressants: a meta-analysis of randomized controlled trials in treating depression. J Med Assoc Thai. 1998; 81:387-392.]. Although the functional influence of these transporter polymorphisms is not fully understood, they are related to the transcription of individual genes. The 1 and s variants of the 5-HTT promoter polymorphism have functional differences in modulating transcription of the 5-HTT gene as well as subsequent 5-HTT availability [reference: Serreti A, Artioli P, Quartesan R. Pharmacogenetics in the treatment of depression: pharmacodynamic studies. Pharmacogenet Genomics. 200515:61-67.]. These allele-specific functional differences have been confirmed in human tissues including the brain [reference: Heils A, Teufel A, Petri S, et al. Allelic variation of human serotonin transporter gene expression. J Neurochem. 1996; 66:2621-2624, and Lesch K P, Bengel D, Heils A, et al. Association of anxiety-related traits with a polymorphism in the serotonin transporter gene regulatory region. Science. 29 1996; 274:1527-1531.]. Thus, the 5-HTT polymorphisms might influence the response to treatment by modulating the transcription of 5-HTT, a direct target of SSRIs.

In the present invention, the patients were mostly the elderly (77% over age 50), and most (60%) had late onset illnesses with few previous depressive episodes. Eighty-eight percent of all cases were in their first or second lifetime episode of depression. The present inventors adopted strict criteria for a previous major depressive episode, excluding minor depression or dysthymia. It is unclear whether late-life depression has distinctive genetic contributions. It is generally accepted that hereditary risk in affective disorder is reduced after 50 years, and that patients with late-onset depression are less likely to have psychiatric co-morbidity and more likely to have medical co-morbidity. However, previous studies have demonstrated that depression symptoms in older adults might be more hereditary than previously thought [reference: Ebmeier K P, Donaghey C, Steele J D. Recent developments and current controversies in depression. Lancet. 2006; 367:153-167], and that early onset and late onset groups do not differ from each other in genotype frequency distribution of the two 5-HTT gene polymorphisms [reference: McGue M, Christensen K. Genetic and environmental contributions to depression symptomatology: evidence from Danish twins 75 years of age and older. J Abnorm Psychol. 1997; 106:439-448, and Golimbet V E, Alfimova M V, Shcherbatykh T V, Rogaev E I. Allele polymorphism of the serotonin transporter gene and clinical heterogeneity of depressive disorders. Genetika. 2002; 38:671-677.]. Likewise, it is unknown whether antidepressant response and pharmacogenetic effects are affected by age or by the age at onset [reference: Steffens D C, Svenson I, Marchuk D A, et al. Allelic differences in the serotonin transporter-linked polymorphic region in geriatric depression. Am J Geriatr Psychiatry. March-April 2002; 10:185-191, and Reynolds C F 3rd, Dew M A, Frank E, et al. Effects of age at onset of first lifetime episode of recurrent major depression on treatment response and illness course in elderly patients. Am J Psychiatry. 1998; 155:795-799.].

The present inventors found no differences of genotype distributions between early onset (onset age of 59 or younger, n=126) and late onset (onset age of 60 or older, n=82) patients (p=0.25, and 0.82 by x² tests for 5-HTTLPR, and 5-HTT intron 2, respectively). The result was similar when the present inventors compared genotype distributions between mid-life (age 59 or younger, n=89) and late-life (age 60 or older, n=119) patients (p=0.30, and 0.35 by x² tests for 5-HTTLPR, and 5-HTT intron 2, respectively). As for treatment response, some previous pharmacogenetic studies reported similar results with elderly and younger patients when controlling for ethnicity and drug [reference: Rausch J L, Johnson M E, Fei Y J, et al. Initial conditions of serotonin transporter kinetics and genotype: influence on SSRI treatment trial outcome. Biol Psychiatry. 2002; 51:723-732, Murphy G M, Jr., Hollander S B, Rodrigues H E, Kremer C, Schatzberg A F. Effects of the serotonin transporter gene promoter polymorphism on mirtazapine and paroxetine efficacy and side effects in geriatric major depression. Arch Gen Psychiatry. 2004; 61:1163-1169, and Reynolds C F, 3rd, Frank E, Kupfer D J, et al. Treatment outcome in recurrent major depression: a post hoc comparison of elderly (“young old”) and midlife patients. Am J Psychiatry. 1996; 153:1288-1292.]. The study of the present invention demonstrates that the responses to antidepressants with different targets have significant associations with gene polymorphisms. The present invention confirmed the association between SSRI responses and polymorphisms of 5-HTT, \textcircled{1}, \textcircled{2}, and \textcircled{3}.

### Advantageous Effects

Through the above described means according to the present invention, it is possible to predict treatment responses to antidepressants according to genetic information, and also to establish customized depression treatment models which are effective in the development of customized new drugs, and appropriate for Korean people. Therefore, the base of domestic clinical trials can be expanded, which will enhance competitive power in the medication market and preoccupy technology of drug prediction.

### BRIEF DESCRIPTION OF DRAWINGS

The foregoing and other objects, features and advantages of the present invention will become more apparent from the following detailed description when taken in conjunction with the accompanying drawing in which:

FIG. 1 shows the performance of clinical trials of SSRI (Selective Serotonin Reuptake Inhibitors) response prediction models using genetic information.

FIGS. 2A and 2B include graphs showing changes in Hamilton depression rating score (HAM-D) over time for various genotypes. The terms used in FIGS. 2A and 2B are as follows: PR: group with combinations of genotypes predicted to response, U: undetermined group, PN: group with combinations of genotypes predicted to nonresponse, OR: Observed response group, and ON: Observed nonresponse group.

### BEST MODE

Hereinafter, the present invention will be described in further detail with reference to examples. It is to be understood, however, that these examples are illustrative only, and the scope of the present invention is not limited thereto.

### EXAMPLES

### Example 1

### Genotype Analysis for VNTR (Variable Number of Tandem Repeat) of 5-HTT Gene

Genomic DNA was extracted from whole blood using a Wizard Genomic DNA Purification kit (Promega, Madison, Wis.). The present inventors analyzed, from patients, the genotype of the 5-HTT promoter s/1 polymorphism (5-HTTLPR), and the genotype of the 5-HTT intron 2 s/1 polymorphism, in order to determine whether or not 5-HTT genotypes known to be the most related to antidepressant treatment responses from conventional researches were reproduced.

5-HTT polymorphism, VNTR polymorphism in the intron 2 region, and 5-HTTLPR (5-HTT-linked polymorphic region) in the promoter region were detected through PCR amplification. For the analysis of VNTR in intron 2 of the serotonin transporter gene, the VNTR region in intron 2 of the serotonin transporter gene containing 17 repeat sequences was amplified by PCR. For this, primers of 8224 (5′-GTCAGTATCACAGGCTGCGAG) (SEQ ID NO: 1) and 8223 (5′-TGTTCCTAGTCTTACGCCAGTG) (SEQ ID NO: 2) were used, and 20 ng genomic DNA, 50 mM KCl, 10 mM Tris.Cl (pH 9.0 at 25° C.), 0.1% Triton-X100, 1 mM MgCl₂, 0.2 mM dNTP, 1 μTaq polymerase, 1 μM sense primer and 1 μM antisense primer were mixed and reacted. The PCR reaction was performed in the following conditions: pre-denaturation at 94° C. for 3 min, and then 25 cycles of denaturation at 94° C. for 30 sec, annealing at 60° C. for 45 sec, and elongation at 72° C. for 45 sec, followed by elongation at 72° C. for 8 min. Then, the temperature was maintained at 4° C. The PCR amplification products were electrophoresed on 3% agarose gel to confirm bands having 9 and 10 copies (s allele) and 12 copies (1 allele), compared to a pUC 18 Hae III digestion marker (Sigma). The 9- and 10-copy VNTR were designated “s allele of 5-HTT intron 2”, and the 12-copy VNTR was designated “1 allele”.

For the analysis of the deletion/insertion polymorphism (5-HTTLPR) of the serotonin transporter gene in the promoter region, PCR reaction was performed using primer stpr5; 5′-GGCGTTGCCGCTCTGAATTGC (SEQ ID NO: 3) corresponding to positions −1,416 to −1,397 of the nucleotide), and primer stpr3; 5′-GAGGGACTGAGCTGGACAACCCAC (SEQ ID NO: 4) corresponding to positions −910 to −889 of the nucleotide). The PCR amplification was performed in a mixture containing 0.1 mM dNTP, 0.15 μM sense and antisense primers, 150 ng genomic DNA, 2 mM Tris.Cl (pH 7.5 at 25° C.), 10 mM KCl, 0.1 mM dithiothreitol (DTT), 0.01 mM EDTA, 0.05% Tween20 (v/v), 0.05% Nonidet P40 (v/v), 5% glycerol, and 1.3 μexpand high fidelity PCR system enzyme mix (Boehringer Mannhein, Mannhein, Germany), in the following conditions: pre-denaturation at 95° C. for 4 min, 10 cycles of denaturation at 95° C. for 30 sec, annealing at 65° C. for 30 sec and elongation at 72° C. for 45 sec, and then 20 cycles of denaturation at 95° C. for 30 sec, annealing at 65° C. for 30 sec and elongation at 72° C. for 4 min and 5 sec, followed by post-elongation at 72° C. for 7 min. Then, the temperature was maintained at 4° C. The amplified products were electrophoresed on 2% agarose gel to confirm bands having 14 copies (s allele), 16, 18, 20 and 22 copies (defined as 1 allele for more than 16 copies), compared to a 100-bp ladder marker. The 14-copy VNTR of 5-HTTLPR was designated “s allele”, and the 16-, 18-, 20- and 22-copy VNTRs were designated “1 alleles”.

### Example 2

### Detection of Blood Drug Level

Blood levels of fluoxetine/norfluoxetine, paroxetine and sertraline were quantified according to conventional methods with liquid chromatography mass spectrometry [reference: Orsulak P J, Liu P K, Akers L C. Antidepressant drugs. In: Shaw L, Ed. The Clinical Toxicology Laboratory. USA, AACC Press; 2001. Tournel G, Houdret N, Hedouin V, Deveau M, Gosset D, Lhermitte M. High-performance liquid chromatographic method to screen and quantitate seven selective serotonin reuptake inhibitors in human serum. J Chromatogr B Biomed Sci Appl. 2001; 761:147-158. Kollroser M, Schober C. Simultaneous determination of seven tricyclic antidepressant drugs in human plasma by direct-injection HPLC-APCI-MS-MS with an ion trap detector. Ther Drug Monit. 2002; 24:537-544.].

### Example 3

### Statistical Analysis

Means and standard deviations (SDs) and ranges of continuous variables, and proportions of categorical variables, are presented as descriptive statistics. The present inventors employed the x² test on categorical variables. Power analyses were performed to examine if the number of patients was sufficient to produce a statistically significant result, given a true difference. Comparisons of the genotype frequencies and allele frequencies between the antidepressant responders and non-responders were performed using Fisher's exact test. A multiple logistic regression model entering all 4 genes was used to evaluate the influence of each gene on the response to the medication by adjusting other genes. Bonferroni's correction was applied to multiple testing. Results were considered significant at P<0.05 after this correction. P-values from Bonferroni's correction were stated with the corrected values. Limited exploratory, post hoc analyses were conducted with Fisher's exact test using a permutation method for multiple testing to examine response rates in relation to genotype combinations. The same method was used to compare differential responses to SSRI by genotype. Measures of linkage disequilibrium (LD) were calculated using the Gold program [reference: Abecasis G R, Cookson W O. GOLD-graphical overview of linkage disequilibrium. Bioinformatics. 2000; 16:182-183.]. All the statistical analyses were performed using SAS software version 9.13 (SAS Institute Inc, Cary, N.C.).

### Example 4

### SNP Analysis Related to the Treatment Response of SSRI Antidepressants

First, 79 candidate genes of monoamine transporter (the primary site of action of antidepressants) and neurotransmitter synthesizing enzyme and receptor, which are related to the antidepressant treatment response, and about 1502 SNPs of the genes were strategically selected, and their genetic information was analyzed on a large scale by Illumina's Golden Gate Assay. Then, the association between 1400 SNPs selected by data quality management and the treatment responses to SSRI antidepressants was analyzed by selecting the most appropriate genotype from 5 genotypes (Dominant, Recessive, Genotype, Allelic, Cochran-Armitage test), and Bonferroni and FDR (False Discovery Rate) were applied to multiple testing. As a result, it was proved that it is possible to treat patients individually by selecting antidepressants showing high treatment success rates. Haplotype blocks were defined by confidence intervals in SSRI treated patients. Association between a haplotype block and response was tested using Fisher's exact test with FDR control. Multivariable analyses for SNPs and for haplotype blocks were performed using multiple logistic regression and GEE (Generalized Estimating Equations) method, respectively. Prediction models were constructed using multiple logistic regression. The probability of response for given genotypic information was computed. We used the operational criteria of probability >0.8 for predicting response (better than the optimal response rate expected with combined drug and cognitive behavioral therapy) and <0.3 for predicting nonresponse (lower than the expected response rate with placebo). We calculated overall accuracy, positive predictive value (PPV), negative predictive value (NPV), sensitivity, specificity with 95% confidence interval, and area under the receiver operating curve (AUC). All P values were reported as two-sided, and P values <0.05 were considered statistically significant. Analyses were performed with the use of the SAS software, version 9.13.

In the following Table 1, 10 SNPs related to the treatment responses of SSRI antidepressants are noted.

TABLE 1

| | | | | Responsive | RAF in | RAF in | |
|---|---|---|---|---|---|---|---|
| Gene | Chromosome | Position* | SNP | Allele | Responders | Nonrespnders | P Value† |
| TPH2 | 12 | 70658496 | rs4760815 | T | 0.60 | 0.41 | 1.26 × 10⁻⁵ |
| TPH2 | 12 | 70663579 | rs11179027 | C | 0.55 | 0.34 | 1.57 × 10⁻⁵ |
| GRIK2 | 6 | 102158042 | rs543196 | C | 0.65 | 0.46 | 4.84 × 10⁻⁵ |
| GAD1 | 2 | 171390986 | rs3828275 | G | 0.72 | 0.64 | 6.89 × 10⁻⁵ |
| TPH2 | 12 | 70650935 | rs17110532 | C | 0.42 | 0.24 | 8.86 × 10⁻⁵ |
| SLC6A4 | 17 | 25575791 | rs2066713 | C | 0.96 | 0.86 | 1.26 × 10⁻⁴ |
| GRIK2 | 6 | 102157181 | rs572487 | G | 0.59 | 0.41 | 1.36 × 10⁻⁴ |
| TPH2 | 12 | 70712221 | rs17110747 | A | 0.31 | 0.16 | 1.94 × 10⁻⁴ |
| GAD1 | 2 | 171379072 | rs12185692 | C | 0.71 | 0.65 | 2.33 × 10⁻⁴ |
| SLC6A4 | 17 | 25571040 | rs2020942 | G | 0.95 | 0.85 | 2.96 × 10⁻⁴ |

| | P Value by | P Value by | | | |
|---|---|---|---|---|---|
| | Bonferroni's | Controlling | | Heterozygote Odds | Homozygote Odds |
| Gene | Correction | FDR | Genetic Mode | Ratio (95% CI) | Ratio (95% CI) |
| TPH2 | 0.02 | 0.02 | Dominant | 3.77 (3.55-4.00) | 4.39 (2.08-9.29) |
| TPH2 | 0.02 | 0.01 | Allele | 2.69 (1.45-4.99) | 4.77 (2.17-10.49) |
| GRIK2 | 0.07 | 0.02 | Additive | 1.69 (0.83-3.45) | 5.02 (2.18-11.53) |
| GAD1 | 0.10 | 0.02 | Genotype | 0.31 (0.17-0.55) | 1.24 (0.43-3.62) |
| TPH2 | 0.12 | 0.02 | Allele | 2.02 (1.14-3.59) | 5.36 (1.93-14.87) |
| SLC6A4 | 0.18 | 0.03 | Recessive | 0.48 (0.03-8.42) | 2.27 (0.14-36.87) |
| GRIK2 | 0.19 | 0.03 | Additive | 1.65 (1.54-1.77) | 4.76 (2.09-10.86) |
| TPH2 | 0.27 | 0.03 | Allele | 2.53 (1.37-4.69) | 3.88 (1.25-11.99) |
| GAD1 | 0.33 | 0.04 | Genotype | 0.35 (0.20-0.62) | 1.57 (0.49-5.03) |
| SLC6A4 | 0.42 | 0.04 | Additive | 1.27 (1.21-1.34) | 4.56 (0.41-51.22) |

| | | | | P Value by | P Value by | | |
|---|---|---|---|---|---|---|---|
| | | | | Bonferroni's | Controlling | | |
| Gene | Chromosome | VNTR | P Value† | Correction | FDR | Genetic Mode | Odds Ratio (95% CI) |
| SLC6A4 | 17 | 5-HTT VNTR in promoter (5-HTTLPR) | 6.00 × 10⁻³ | NA | NA | ss vs. sl + ll | 2.18 (1.27-3.75) |
| SLC6A4 | 17 | 5-HTT VNTR in intron 2 (STin2) | 2.00 × 10⁻⁴ | NA | NA | ll vs. sl + ss | 3.86 (1.90-7.84) |

The terms used in Table 1 are explained as follows: RAF: responsive allele frequency, FDR: false discovery rate, VNTR: variable number of tandem repeat, NA: not applicable, *: genetic identity (NCBI Build 36), †: Fisher's exact test.

In the present invention, in order to develop a means for predicting the treatment response of an antidepressant by using genotypes, an antidepressant treatment response predicting means was obtained by the combination of results of 10 SNPs showing significant association with 6 haplotypes. In other words, as noted in Tables 2 to 6, 4 antidepressant treatment response predicting models were built by combining genotypes of TPH2 (serotonin biosynthesizing enzyme), SLC6A4 (serotonin transporter, 5-HTT), GAD1 (GABA biosynthesizing enzyme), and GR1K2 (glutamate receptor).

TABLE 2
1) Polymorphism Model

| | | | | | | Predictability of |
|---|---|---|---|---|---|---|
| | rs4760815 | rs543196 | RS3828275 | rs2066713 | 5-HTTLPR | response (%) |
| PR | AT + TT | CC | AA | CC | ss | 95.8 |
| | AT + TT | CC | GG | CC | ss | 95 |
| | AT + TT | CC | AA | CC | sl + ll | 90.9 |
| | AT + TT | TC | AA | CC | ss | 90.4 |
| | AT + TT | CC | GG | CC | sl + ll | 89.4 |
| | AT + TT | TC | GG | CC | ss | 88.9 |
| | AT + TT | CC | AG | CC | ss | 84.7 |
| | AT + TT | CC | AA | TC + TT | ss | 83.5 |
| | AA | CC | AA | CC | ss | 81.3 |
| | AT + TT | CC | GG | TC + TT | ss | 81 |
| | AT + TT | TC | AA | CC | sl + ll | 80.7 |
| PN | AA | CC | AA | TC + TT | sl + ll | 30 |
| | AT + TT | TT | AG | CC | sl + ll | 29.9 |
| | AA | TC | AA | TC + TT | ss | 28.9 |
| | AT + TT | TT | AA | TC + TT | sl + ll | 28 |
| | AA | CC | GG | TC + TT | sl + ll | 26.6 |
| | AA | TC | GG | TC + TT | ss | 25.5 |
| | AA | TT | AA | CC | sl + ll | 25.2 |
| | AT + TT | TT | GG | TC + TT | sl + ll | 24.7 |
| | AA | TT | GG | CC | sl + ll | 22.1 |
| | AA | CC | AG | TC + TT | ss | 19.2 |
| | AT + TT | TC | AG | TC + TT | sl + ll | 18.6 |
| | AT + TT | TT | AG | TC + TT | ss | 17.8 |
| | AA | TC | AG | CC | sl + ll | 16.5 |
| | AA | TT | AG | CC | ss | 15.7 |
| | AA | TC | AA | TC + TT | sl + ll | 15.2 |
| | AA | TT | AA | TC + TT | ss | 14.5 |
| | AA | TC | GG | TC + TT | sl + ll | 13.1 |
| | AA | TT | GG | TC + TT | ss | 12.6 |
| | AA | CC | AG | TC + TT | sl + ll | 9.5 |
| | AA | TC | AG | TC + TT | ss | 9.1 |
| | AT + TT | TT | AG | TC + TT | sl + ll | 8.7 |
| | AA | TT | AG | CC | sl + ll | 7.6 |
| | AA | TT | AA | TC + TT | sl + ll | 7 |
| | AA | TT | GG | TC + TT | sl + ll | 5.9 |
| | AA | TC | AG | TC + TT | sl + ll | 4.2 |
| | AA | TT | AG | TC + TT | ss | 4 |
| | AA | TT | AG | TC + TT | sl + ll | 1.8 |

TABLE 3
2) Polymorphism Simpler Model

| rs4760815 | rs2066713 | Predictability of response (%) |
|---|---|---|
| AT + TT | CC | 78 |
| AA | CC | 42.6 |
| AT + TT | TC + TT | 38.2 |
| AA | TC + TT | 11.5 |

TABLE 4
3) Haplotype Model

| | TPH2 | SLC6A4 | | | | |
|---|---|---|---|---|---|---|
| | (H3)* | (H1)† | rs543196 | rs3828275 | 5-HTTLPR | % |
| PR | H3-B | H1-A | CC | AA | ss | 95.2 |
| | H3-B | H1-A | CC | GG | ss | 93.6 |
| | H3-B | H1-A | CC | AA | sl + ll | 90.9 |
| | H3-B | H1-A | TC | AA | ss | 89.5 |
| | H3-B | H1-A | CC | GG | sl + ll | 88 |
| | H3-B | H1-A | TC | GG | ss | 86.2 |
| | H3-B | H1-A | CC | AG | ss | 85.2 |
| | H3-B | H1-A | TC | AA | sl + ll | 81 |
| PN | H3-B | H1-B | TT | GG | ss | 30 |
| | H3-B | H1-B | TC | AG | ss | 28.2 |
| | H3-A | H1-A | TT | AA | ss | 27.7 |
| | H3-A | H1-B | CC | AA | ss | 24.9 |
| | H3-A | H1-A | TC | GG | sl + ll | 24.8 |
| | H3-A | H1-A | CC | AG | sl + ll | 23.3 |
| | H3-B | H1-B | TT | AA | sl + ll | 22.6 |
| | H3-A | H1-A | TT | GG | ss | 22 |
| | H3-A | H1-A | TC | AG | ss | 20.6 |
| | H3-A | H1-B | CC | GG | ss | 19.6 |
| | H3-B | H1-B | TT | GG | sl + ll | 17.7 |
| | H3-B | H1-B | TC | AG | sl + ll | 16.5 |
| | H3-A | H1-A | TT | AA | sl + ll | 16.1 |
| | H3-B | H1-B | TT | AG | ss | 14.4 |
| | H3-A | H1-B | CC | AA | sl + ll | 14.3 |
| | H3-A | H1-B | TC | AA | ss | 12.5 |
| | H3-A | H1-A | TT | GG | sl + ll | 12.4 |
| | H3-A | H1-A | TC | AG | sl + ll | 11.5 |
| | H3-A | H1-B | CC | GG | sl + ll | 10.9 |
| | H3-A | H1-A | TT | AG | ss | 10 |
| | H3-A | H1-B | TC | GG | ss | 9.5 |
| | H3-A | H1-B | CC | AG | ss | 8.8 |
| | H3-B | H1-B | TT | AG | sl + ll | 7.8 |
| | H3-A | H1-B | TC | AA | sl + ll | 6.7 |
| | H3-A | H1-B | TT | AA | ss | 5.8 |
| | H3-A | H1-A | TT | AG | sl + ll | 5.3 |
| | H3-A | H1-B | TC | AA | sl + ll | 5 |
| | H3-A | H1-B | CC | AG | sl + ll | 4.6 |
| | H3-A | H1-B | TT | GG | ss | 4.3 |
| | H3-A | H1-B | TC | AG | ss | 4 |
| | H3-A | H1-B | TT | AA | sl + ll | 3 |
| | H3-A | H1-B | TT | GG | sl + ll | 2.2 |
| | H3-A | H1-B | TC | AG | sl + ll | 2 |
| | H3-A | H1-B | TT | AG | ss | 1.7 |
| | H3-A | H1-B | TT | AG | sl + ll | 0.9 |

TABLE 5
4) Haplotype Simpler Model

| TPH2 | SLC6A4 | Predictability of |
|---|---|---|
| (H3)* | (H1)† | response (%) |
| H3-B | H1-A | 78.4 |
| H3-B | H1-B | 31.8 |
| H3-A | H1-A | 23.6 |
| H3-A | H1-B | 3.8 |

The terms used in Tables 2 to 5 are explained as follows: PR: Predicted Responder, PN: Predicted Nonresponder, *: H3-A is defined by two haplotype sets (GCATGG and GCATGG), and H3-B is defined by 65 haplotype sets (GCATGG and ACGTGT; GCATGG and ATGTAT; GCATGG and ATGTGT; GCATGG and GCACGG; GCATGG and GCACGT; GCATGG and GCATAG; GCATGG and GCGTGT; GCATGG and GTGTAT; GCATGG and GTGTGG; GCATGG and GTGTGT; GCATAG and GCATAG; GCATAG and GCACGG; GCATAG and GCACGT; GCATAG and GCGTGT; GCATAG and GTGTGG; GCATAG and ACGTGT; GCATAG and GTGTGT; GCATAG and ATGTAT; GCATAG and GTGTAT; GCATAG and ATGTGT; GCACGG and GCACGG; GCACGG and GCACGT; GCACGG and GCGTGT; GCACGG and GTGTGG; GCACGG and ACGTGT; GCACGG and GTGTGT; GCACGG and ATGTAT; GCACGG and GTGTAT; GCACGG and ATGTGT; GCACGT and GCACGT; GCACGT and GCGTGT; GCACGT and GTGTGG; GCACGT and ACGTGT; GCACGT and GTGTGT; GCACGT and ATGTAT; GCACGT and GTGTAT; GCACGT and ATGTGT; GCGTGT and GCGTGT; GCGTGT and GTGTGG; GCGTGT and ACGTGT; GCGTGT and GTGTGT; GCGTGT and ATGTAT; GCGTGT and GTGTAT; GCGTGT and ATGTGT; GTGTGG and GTGTGG; GTGTGG and ACGTGT; GTGTGG and GTGTGT; GTGTGG and ATGTAT; GTGTGG and GTGTAT; GTGTGG and ATGTGT; ACGTGT and ACGTGT; ACGTGT and GTGTGT; ACGTGT and ATGTAT; ACGTGT and GTGTAT; ACGTGT and ATGTGT; GTGTGT and GTGTGT; GTGTGT and ATGTAT; GTGTGT and GTGTAT; GTGTGT and ATGTGT; ATGTAT and ATGTAT; ATGTAT and GTGTAT; ATGTAT and ATGTGT; GTGTAT and GTGTAT; GTGTAT and ATGTGT; and ATGTGT and ATGTGT). †: H1-A is defined by a certain set including any one haplotype selected from haplotypes, such as CATAGGGATGCC, CATAGGGACGCC, CATAGGAACGTC, CCTAGGGATGCC, AATAGGGATGCC, AACGAGGCCCCT, AACGAGAATGCC and AACGAAGCCCCT (SEQ ID NO: 5 to SEQ ID NO: 12), and H1-B is defined by a certain set including any one haplotype selected from haplotypes, such as AACGAGAACGTC, CATAGGGCCCCC and CATGAGGATGCC (SEQ ID NO: 13 to SEQ ID NO: 15).

Tables 2 to 5 show pharmacogenomic-based results: genotypes of 1) Polymorphism Model and 3) Haplotype Model, and genotypes of 2) Polymorphism Simpler Model and 4) Haplotype Simpler Model.

As noted in Tables 2 to 5, for SNPs within 4 genes showing the most significant association with treatment responses to SSRI antidepressants, in LD (Linkage Disequilibrium) block, the general association between the treatment responses to antidepressants and various SNPs was observed according to haplotypes. As a result, it was determined that 6 haplotypes are significant. H3, H4, and H5 of TPH2, H8 and H9 of GRIK2, and H1 of SERT showed significant association with treatment responses to SSRI antidepressants.

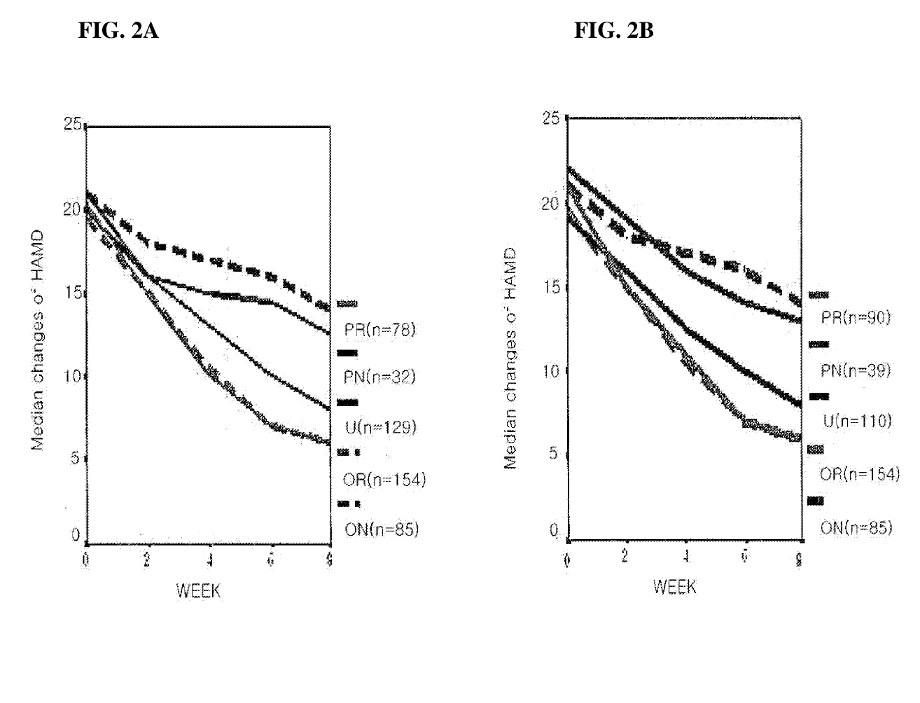

Also, the following Table 6 (and FIGS. 2A and 2B) shows the prediction of the antidepressant treatment responses of 4 antidepressant treatment response prediction models by using genotype information. The prediction accuracy of these models (pharmacogenomic models) is the highest, compared to other antidepressant treatment response prediction models. Also, polymorphism models including rs4760815 of TPH2, 5-HTTLPR and rs2066713 of SLC6A4, rs3828275 of GAD1, and rs543196 of GRIK2 showed a high treatment success prediction rate of PPV (Positive Predictive Value) 0.90, and NPV (Negative Predictive Value) 0.88 (sensitivity: 0.45, and specificity: 0.33, see Table 6).

TABLE 6

| | | 5-HTTLPR, |
|---|---|---|
| Information | 5-HTTLPR, 4 SNPs | 2 SNPs (rs3828275, |
| of | (rs3828275, rs543196, | rs543196), 2 haplotype |
| genotypes | rs2066713, rs4760815) | blocks (SLC6A4, TPH2) |
| PPV (%: 95% | 70/78 (0.90: 0.83, | 79/90 (0.88: 0.81, 0.95) |
| CI) | 0.96) | |
| Sensitivity | 70/154 (0.45: 0.38, | 79/154 (0.51: 0.43, 0.59) |
| (%: 95% CI) | 0.53) | |
| NPV (%: 95% | 28/32 (0.88: 0.76, | 33/39 (0.85: 0.73, 0.96) |
| CI) | 0.99) | |
| specificity | 28/85 (0.33: 0.23, | 33/85 (0.39: 0.28, 0.49) |
| (%: 95% CI) | 0.43) | |
| AUC | 0.81 | 0.78 |
| Short form | rs2066713, rs4760815 | SLC6A4, TPH2 |
| PPV (%: 95% | 122/156 (0.78: 0.72, | 137/175 (0.78: 0.72, |
| CI) | 0.85) | 0.84) |
| sensitivity | 122/154 (0.79: 0.73, | 136/154 (0.89: 0.84, |
| | 0.86) | 0.94) |
| NPV | 51/83 (0.61: 0.51, | 47/64 (0.73: 0.63, 0.84) |
| | 0.72) | |
| specificity | 51/85 (0.60: 0.50, | 47/85 (0.55: 0.45, 0.66) |
| | 0.70) | |
| AUC | 0.7 | 0.73 |

The terms used in Table 6 are explained as follows: PPV: positive predictive value, NPV: Negative predictive value, AUC: Area under the ROC curve, PR: group with combinations of genotypes predicted to response, U: undetermined group, PN: group with combinations of genotypes predicted to nonresponse, OR: Observed response group, and ON: Observed nonresponse group.

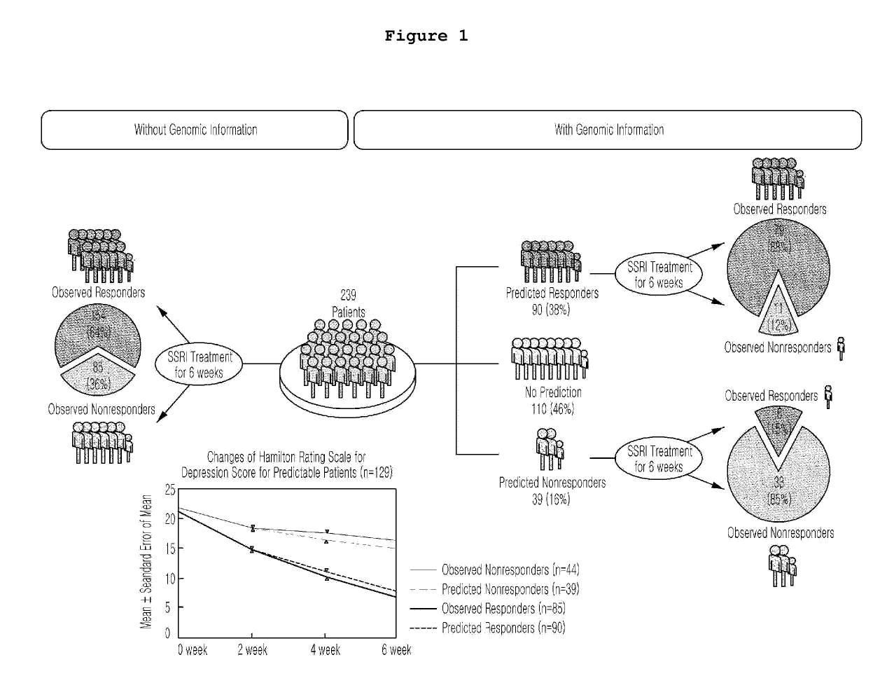

Meanwhile, FIG. 1 shows the performance of clinical trials of SSRI (Selective Serotonin Reuptake Inhibitors) response prediction models using genetic information. In other words, based on haplotype models, the association with genetic information was determined by changes of HAM-D (Hamilton depression rating score) and response rates for 6 weeks after SSRI-treatment (see FIG. 1 and also FIGS. 2A and 2B). This is for the most genotype markers in all antidepressant pharmacogenomic researches reported so far. Through the research, the treatment success rate of SSRI antidepressants was estimated to be 88% at the maximum, while conventional research showed 50 to 60% (see FIG. 1).

### Example 5

### A Kit and Biomarker for Predicting the Therapeutic Effects of SSRI Antidepressants

In the present invention, a kit and a biomarker for predicting the therapeutic effects of SSRI antidepressants by using 4 prediction models noted in Tables 2 to 6 (and FIGS. 2A and 2B) were obtained. In other words, (1) a kit and biomarker including primers for screening 5-HTTLPR and 4 SNPs (rs3828275, rs543196, rs2066713, and rs4760815), genotyping primers, and a probe, (2) a kit and biomarker including primers for screening 5-HTTLPR, 2 SNPs (rs3828275, and rs543196), and 2 haplotype blocks (SLC6A4, and TPH2), genotyping primers, and a probe, (3) a kit and biomarker including primers for screening 2 SNPs (rs2066713, and rs4760815), genotyping primers, and a probe, and (4) a kit and biomarker including primers for screening 2 haplotype blocks (SLC6A4, and TPH2), genotyping primers, and a probe were obtained.

1-12. (canceled)

13. A method for providing information on a therapeutic effect of an SSRI antidepressant comprising detecting the presence of or the absence of a Tryptophan Hydroxylase 2 (TPH2) gene polymorphism rs4760815 or TPH2 H3 haplotype; a Solute Carrier Family 6 Member 4 (SLC6A4) gene polymorphism 5-HTTLPR, SLC6A4 H1 haplotype, or SLC6A4 gene polymorphism rs2066713; a Glutamate Decarboxylase) (GAD1) gene polymorphism rs3828275; or a Glutamate Receptor Ionotropic kainate 2 (GRIK2) gene polymorphism rs543196.

14. A kit for predicting a therapeutic effect of an SSRI antidepressant, the kit comprising primers for screening a Tryptophan Hydroxylase 2 (TPH2) gene polymorphism rs4760815 or TPH2 H3 haplotype; a Solute Carrier Family 6 Member 4 (SLC6A4) gene polymorphism 5-HTTLPR, SLC6A4 H1 haplotype, or SLC6A4 gene polymorphism rs2066713; a Glutamate Decarboxylase1 (GAD1) gene polymorphism rs3828275; or a Glutamate Receptor Ionotropic kainate 2 (GRIK2) gene polymorphism rs543196, genotyping primers, and a probe.

15. A biomarker for predicting a therapeutic effect of an SSRI antidepressant, the biomarker comprising a Tryptophan Hydroxylase 2 (TPH2) gene polymorphism rs4760815 or TPH2 H3 haplotype; a Solute Carrier Family 6 Member 4 (SLC6A4) gene polymorphism 5-HTTLPR, SLC6A4 H1 haplotype, or SLC6A4 gene polymorphism rs2066713; a Glutamate Decarboxylase1 (GAD1) gene polymorphism rs3828275; or a Glutamate Receptor Ionotropic kainate 2 (GRIK2) gene polymorphism rs543196.
